(19) United States
(12) Patent Application Publication
(10) Pub. No.: US 2003/0165842 A1
(43) Pub. Date: Sep. 4, 2003

(54) Method for identifying biological binding molecules and apparatus for carrying out the method

(76) Inventors: Jocelyn Ng, Munchen (DE); Daniel G. Jay, Brighton, MA (US); Liming Ge, Munchen (DE); Leodevico L. llag, Munchen (DE)

Correspondence Address: CLARK & ELBING LLP, 101 FEDERAL STREET, BOSTON, MA, 02110, US

(21) Appl. No.: 09/908,100

(22) Filed: Jul. 18, 2001

Related U.S. Application Data
(63) Continuation-in-part of application No. 09/444,959, filed on Nov. 22, 1999, now abandoned.

(30) Foreign Application Priority Data
Nov. 24, 1998 (DE) 19854195.3

(51) Int. Cl. C12Q 1/68

(52) U.S. Cl. 435/6

(57) ABSTRACT

The invention relates to a method for identifying the function of a ligand L using chromophore-assisted laser inactivation (CALI), characterized by the stages:

a) selecting a ligand binding partner (LBP) with specificity for the ligand L,

b) coupling the LBP to a laser-activatable marker (tag) to form LBP-tag, where appropriate after previous modification of the LBP with the aim of more efficient binding to the marker,

c) bringing the ligand L into contact with at least one LBP-tag to form an L/LBP-tag complex, and

d) irradiating the L/LBP-tag complex with a laser beam, whereupon the irradiated LBP-tag selectively modifies the bound ligand, it being possible to interchange the sequence of stages b) and c).

The invention also relates to an apparatus for carrying out the method according to the invention.

### CROSS-REFERENCES TO RELATED APPLICATIONS

This application claims the benefit of U.S. application U.S. Ser. No. 09/444,959, filed Nov. 22, 1999, and foreign patent application DE 198 54 195.3, filed Nov. 24, 1998 in Germany.

### FIELD OF THE INVENTION

The invention relates to a method and an apparatus for identifying the function of biological molecules. In particular, the invention relates to a method for identifying the function of biological molecules using chromophore-assisted laser inactivation (CALI). The method is based on the specific binding of a ligand to its binding partner and inactivation thereof by the CALI method.

The present invention relates to the area of functional genomics and, in particular, it relates to a method using ligand binding partners (LBP), preferably selected from combinatorial libraries or produced by genetic manipulation, in order to modify a target ligand (L) in order to determine the function of the target ligand.

### BACKGROUND OF THE INVENTION

The expansion of the field of genomics has rapidly lead to the identification of complete DNA sequences of organisms. It is assumed that in the year 2003 the entire human genome will be completely sequenced and the complete structural information will be decoded. However, knowledge of the DNA sequence on its own does not provide sufficient information for understanding how genes and diseases correlate in order to make it possible to develop more effective therapies of diseases. The plethora of information has led to the development of new technologies in order to understand the function of these genes better. However, at present, this is not possible.

The CALI method relates to the direct inactivation of a protein. The CALI method is the most promising method for determining the function of a protein. It is not always possible with gene knockouts, for example, to obtain mutations for a specific protein, and many animal systems are not readily amenable to genetic investigations. In the other methods, inhibiting pharmaceuticals (ribozymes and antisenses) can be used only for a restricted number of proteins, and function-blocking antibodies and aptamers represent only a small portion of those which are produced. On the other hand, CALI can convert “binding reagents” such as antibodies or ligands into function blockers. Briefly, a probe (ligand binding partner, LBP) is labelled with the dye malachite green (MG) or another chromophore which generates free radicals after exposure to laser light of a wavelength which is not significantly absorbed by the cellular components. The MG-labelled LBP (LBP-MG) is incubated with the sample of interest. An inactivation region is selected and irradiated with a laser beam at 620 nm. The light is absorbed by MG, generating short-lived free radicals which selectively inactivate proteins bound to LBP-MG within a radius of 15 Å. The system is versatile because it can be used for in vitro and in vivo assays and for intracellular and extracellular target molecules.

CALI represents a promising tool for throwing light on the function of genes, but in the state of the art CALI is restricted to the use of whole antibodies (Jay, D. G. 1988, PNAS 85, 5454-5458) and Fab molecules (Surrey, T. et al. 1998, PNAS 95, 4293-4298). It has been proposed that antibody molecules can be generated from the screening of monoclonal libraries or hybridomas (Wang, F. -S. & Jay, D. G. 1996, Trends in Cell Biology 6, 442-445). However, this variant is extremely time-consuming. Another restriction is that the whole antibody molecules or Fabs are relatively large and therefore CALI possibly does not inactivate the protein in cases where the distance between the MG binding sites in the antibody molecule and the domains required for the function of the protein is large, or the sensitivity of the domain to damage by hydroxyl free radicals is low. Although it has been proposed that CALI can be used with every LBP, there are as yet no possible uses thereof.

Improvements for CALI have already been proposed (Surrey, T. et al. 1998, PNAS 95, 4293-4298). It has been shown that the fluorescein molecule can be used as chromophore in place of malachite green. Fluorescein has the advantage that it is more soluble and more efficient at inactivation, and it is possible to use a more suitable light source with a continuous wavelength. It has also been shown that the Fab fragment is functional in CALI experiments. However, CALI has been modified so that one epitope (i.e. haemagglutinin (HA)) is adapted to each target molecule of interest and subsequent CALI experiments can be carried out. Thus, only one Fab fragment, (i.e. anti-HA Fab) serves as ligand for all target molecules which are to be inactivated by CALI.

Although this method is useful for investigations in which no non-inactivating ligands which can be used for controlled investigations are available, it has several restrictions on use in the field of functional genomics or the assessment of the target molecule. In the first place, the method is very complicated because every target molecule must be manipulated so that it includes the specific epitope. Furthermore, the introduced epitope may break up the function of the target molecule, which will make it necessary to introduce a site which is better tolerated into the molecule. However, if this site which is better tolerated is too remote from the functional site in the molecule to be investigated and is located outside the inactivation region, CALI does not work. In the second place, although investigations have shown that Fab can be used for CALI, this cannot be generalized because antibodies are intrinsically variable. It may occur that the chromophore had a favourable attachment site in the described anti-HA Fab and this does not apply to another Fab. Hence there is a need to establish where the chromophore binds to the anti-HA Fab and to manipulate the latter, viz. this position, for a generalization to other Fabs. Alternatively, ligands smaller than Fabs may be required to make CALI more efficient. Although the investigations further made it obvious to use smaller molecules for CALI, it was also confined to the same molecular environment as this application. It is therefore subject to the same restrictions which must be overcome for CALI to be an effective tool for functional genomics and assessment of the target molecule.

A further restriction on CALI as method for inactivating intracellular target molecules is the process of delivering the labelled ligand to the cell. Although existing methods, such as trituration and microinjection, have functioned, this might be restricted to certain cell types. Very recent developments in electroporation may likewise be used (Rols, M.-O. et al. 1998, Nature Biotechnology 16, 168-171).

The present invention is therefore based on the object of modifying or eliminating current restrictions on the CALI method. It is intended to provide novel methods which improve the efficiency and speed of carrying out CALI. It is likewise intended that the method be applicable to all binding partners and be automatable. The method is intended to permit simple determination of the function of any molecules.

### BRIEF SUMMARY OF THE INVENTION

The present invention therefore relates to a method for identifying the function of a ligand L using chromophore-assisted laser inactivation (CALI), characterized by the stages:

a) selecting a ligand binding partner (LBP) with specificity for the ligand L,

b) coupling the LBP to a laser-activatable marker (tag) to form LBP-tag, where appropriate after previous modification of the LBP with the aim of more efficient binding to the marker,

c) bringing the ligand L into contact with at least one LBP-tag to form an L/LBP-tag complex, and

d) irradiating the L/LBP-tag complex with a laser beam, whereupon the irradiated LBP-tag selectively modifies the bound ligand, it being possible to interchange the sequence of stages b) and c).

### BRIEF DESCRIPTION OF THE DRAWINGS

FIG. 1 is a diagrammatic representation of the functional identification of biological molecules.

FIG. 2 is a schematic diagram of an apparatus for carrying out CALI.

FIG. 3 is a bar graph showing the binding of unlabeled and FITC-labeled anti-β-galactosidase scFv antibodies measured by ELISA, wherein: (1) unselected controls; (2) scFv pool after one round of anti-β-galactosidase; (3) non-binding scFv 1A1 (negative control); (4) anti-β-galactosidase scFv 1A4; (5) pool of 20 anti-β-galactosidase scFv; (6) anti-β-galactosidase scFv 3A6. (a) and (b) represent scFv used at 1 μg/ml on wells coated with 50 nM β-galactosidase or 2.5% low-fat milk, respectively; and (c) and (d) represent scFv used at 0.1 μg/ml on wells coated with 50 nM β-galactosidase or 2.5% low-fat milk, respectively.

FIG. 4 is a bar graph of the CALI of β-galactosidase using FITC-labeled anti-β-galactosidase scFv and whole antibodies, wherein: (1) unselected controls; (2) scFv pool after one round of anti-β-galactosidase; (3) non-binding scFv 1A1 (negative control); (4) anti-β--galactosidase scFv 1A4; (5) pool of 20 anti-β-galactosidase scFv; (6) anti-β-galactosidase scFv 3A6; (7) polyclonal rabbit anti-β-galactosidase IgG; (8) non-immune rabbit IgG. (a) represents non irradiated samples and (b) are samples irradiated for 5 minutes at 494 nm.

FIG. 5 is a bar graph showing the binding, measured by ELISA, of an unlabeled and FITC-labeled scFv antibody and its diabody version containing a lysine-rich linker, wherein: (1) unlabeled scFv; (2) FITC-labeled scFv 1A4; (3) unlabeled, lysine-rich diabody 1A4; and (4) RITC-labeled, lysine-rich diabody 1A4; where (a) and (b) are binding reactions performed in wells coated with 50 nM β-galactosidase, or 2.5% low-fat milk, respectively.

### DETAILED DESCRIPTION OF THE INVENTION

It has been found, surprisingly, that a combination of the CALI method with suitable methods for identifying the ligand permits rapid and simple determination of the function of a molecule. It has previously been assumed that the CALI method can be used only for known ligands with predetermined specificities such as, for example, enzyme substrates. It was not previously possible to use CALI in cases where no known ligands were available. It was further previously impossible to combine CALI with the screening of combinatorial libraries.

CALI has now been combined according to the invention with the screening of combinatorial libraries for the first time. It has been shown according to the invention that it is possible to label a LBP at a specific site deliberately with a chromophore, which markedly increases the possibilities for the CALI method. The method can be used to identify the function of any molecules such as, for example, proteins, peptides, carbohydrates, lipids, DNA, RNA etc.

Firstly, according to the invention an LBP with specificity for the ligand is selected.

At least one selected LBP is coupled to a marker, preferably a laser-activated marker, to form an LBP-tag. The coupling of LBP to the marker can also take place after complex formation. Then L is brought into contact with the LBP-tag to form an L/LBP-tag complex. The L/LBP-tag complex is brought into contact with an inducer, preferably this is done by irradiation with a laser beam, whereby the tag leads to the selective modification of the bound ligand (L).

The LBP is any molecule suitable for binding to a ligand. It is preferably selected from the following molecules: scFv, Fab, diabody, immunoglobulin-like molecules, peptide, RNA, DNA, PNA and small organic molecules except intact antibody molecules. In another preferred embodiment, the aforementioned LBPs are selected from a combinatorial library by one of the following methods, which are known to a skilled person: one-stage selection, (cf. DE 19802576.9), phage display (Cwirla, S. E. et al. 1997, Science 276, 1696-1699), peptides on a plasmid (Stricker, N. L. et al. 1997, Nature Biotechnology 15, 336-342), SIP (Spada, S. et al. 1997, Biol. Chem. 378, 445-456), CLAP (Malmborg, A. -C. et al. 1997, JMB 273, 544-551), ribosome/polysome display (Kawasaki, G. 1991, international patent application WO 91/05058; Hanes, J. & Pluckthun, A. 1997, PNAS 94, 4937-4942) or systematic evolution of ligands by exponential enrichment (SELEX; Tuerk, C. & Gold, L. 1990, Science 249, 505-510).

The binding partner is preferably derived from a combinatorial library. This can be any suitable combinatorial library, for example protein library, peptide library, cDNA library, mRNA library, library with organic molecules, scFv library with immunoglobulin superfamily, protein display library etc. The following may be represented in the libraries: all types of proteins, for example structural proteins, enzymes, receptors, ligands, all types of peptides including modifications, DNAs, RNAs, combinations of DNAs and RNAs, hybrids of peptides and RNA or DNA, all types of organic molecules, for example steroids, alkaloids, natural products, synthetic substances etc. The presentation can take place in various ways, for example as phage display system (for example filamentous phages such as M13, fl, fd etc., lambda phage display, viral display etc.), presentation on bacterial surfaces, ribosomes etc.

The activatable marker can be any suitable molecule which can be linked covalently or noncovalently to a LBP and is able to produce free radicals after induction. Examples thereof are photoinducible molecules, for example peroxidase with hydrogen peroxide and laser-activatable markers. The latter are preferably used.

In a preferred embodiment, the laser-activatable marker is malachite green, fluorescein, lissamine rhodamine, tetramethylrhodamine isothiocyanate, cyanin 3.18.

Preference is furthermore given to AMCA-SE (7-amino-4-methylcoumarin-3-acetic acid, succinimidyl ester), AMCA (7-amino-4-methylcoumarin-3-acetic acid), BODIPY® (4,4-difluoro-4-bora-3a,4a-diaza-S-indacene) and variants thereof, Cascade Blue, Cl-NERF, dansyl, dialkylaminocoumarin, 4′,5′-dichloro-2′,7′-di-methyoxyfluorescein, DM-NERF, eosin, eosin F3S, erythrosin, hydroxycoumarin, Isosulfan Blue, lissamine rhodamine B, methoxycoumarin, naphthofluorescein, NBD, Oregon Green 488, 500, 514, PyMPO (4-(5-(4-methoxyphenyl)oxazol-2-yl)pyridine), pyrene, Rhodamine 6G, Rhodamine Green, Rhodamine Red, Rhodol Green, 2′,4′,5′,7′-tetrabromo-sulphonefluorescein, tetramethylrhodamine, Texas Red or X-rhodamine. The irradiation takes place with laser light of a wavelength which is absorbed by the particular chromophore.

In a preferred embodiment, the selected LBP or every preexisting LBP is modified so that the efficiency of coupling or binding of the marker is increased, forming a more efficient or more stable LBP-tag molecule.

For example, lysine residues can be added onto the protein, making it easier to couple dye molecules such as malachite green or fluorescein via isothiocyanate linkers. It is also possible to produce bispecific molecules by methods known per se, with one specificity being directed, for example, against a chromophore and the other against the ligand.

One variant is to produce a bispecific molecule with specificities both for the ligand and for the marker. Firstly an scFv or LBP specific for the tag is selected by means of phage display, peptides on a plasmid, SIP, CLAP, ribosome/polysome display or SELEX (systematic evolution of ligands by exponential enrichment). This specific LBP for the marker can then be coupled to a second domain with specificity for the target ligand (L), producing a molecule with two specificities. This can be carried out using methods which are known to a person skilled in the field, such as, for example, by means of diabodies (Holliger, P. et al. 1993, PNAS 90, 6444-6448) through the use of a domain association such as Fos-Jun leucine zipper (O'Shea, E. K. et al. 1989, Science 245, 645-648; Kostenly, S. A. et al. 1992, J. Immunol. 148, 1547-1553), single-chain chimeric molecules with two specificities (Gruber M. et al. 1994, J. Immunology 152, 5368-5374; Mack, M. et al. 1995, PNAS 92, 7021-7025) or chimeric SELEX (systematic evolution of ligands by exponential enrichment). It is further possible to produce LBP by genetic manipulation so that it contains amino acids (for example Cys and Lys) which are readily amenable to chemical linkage to the chromophore (Hudson, L. & Hay, F. C. 1976, Fluorescein Conjugation technique. In Practical Immunology. Blackwell Scientific Publications, Boston, chapter 2.1.1, 14-17; Jay, D. G. 1988, PNAS 85, 5454-5458; Surrey, T. et al. 1998, PNAS 95, 4293-4298). Compounds obtainable commercially are used in these cases.

It is possible in a preferred embodiment if an LBP is a whole antibody molecule to convert the latter into an scFv or Fab by recombinant methods known to every person skilled in the field (Clackson, T. et al. 1991, Nature 352, 624-628; Huston, J. S. et al. 1988, PNAS 85, 5879-5883).

In another embodiment, the LBP undergoes genetic manipulation so that it contains amino acid sequences which allow efficient transport of LBP into the cells in order to make CALI more widely applicable to a wide spectrum of cell types and increase the efficiency for intracellular target molecules. It has been shown that short peptide sequences (Rojas, M. et al, 1998, Nature Biotechnology 16, 370-375) and the herpesvirus VP22 protein (Phelan, A. et al. 1998, Nature Biotechnology 16, 440-443) fused to heterologous proteins lead to efficient transport into mammalian cells. Thus, for example, signal peptide sequences or homeodomains which facilitate the transport of large protein molecules into the cells can be linked to the desired LBPs by genetic engineering fusion or by a chemical coupling method. Methodological steps of this type are well known to a person skilled in this field. Methods appropriate for chemical coupling are likewise known to a skilled person, for example coupling via SH-protected cysteins.

The ligand and the LBP-tag are brought into contact under conditions under which complex formation takes place. Conditions of this type have already been investigated during research into ligand interactions, mobility tests etc. and are well known to a skilled person. These are preferably physiological conditions. The complex formation between the ligand and the labelled binding partner takes place very specifically and is not impaired by concomitant processes or complexes.

The modified ligand is identified using a suitable chemical/physical method.

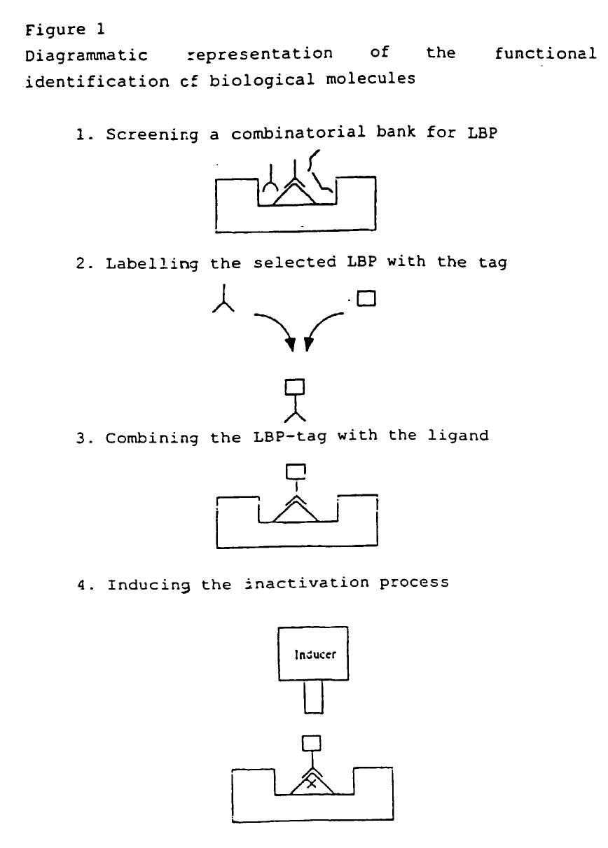

FIG. 1 outlines the method according to the invention.

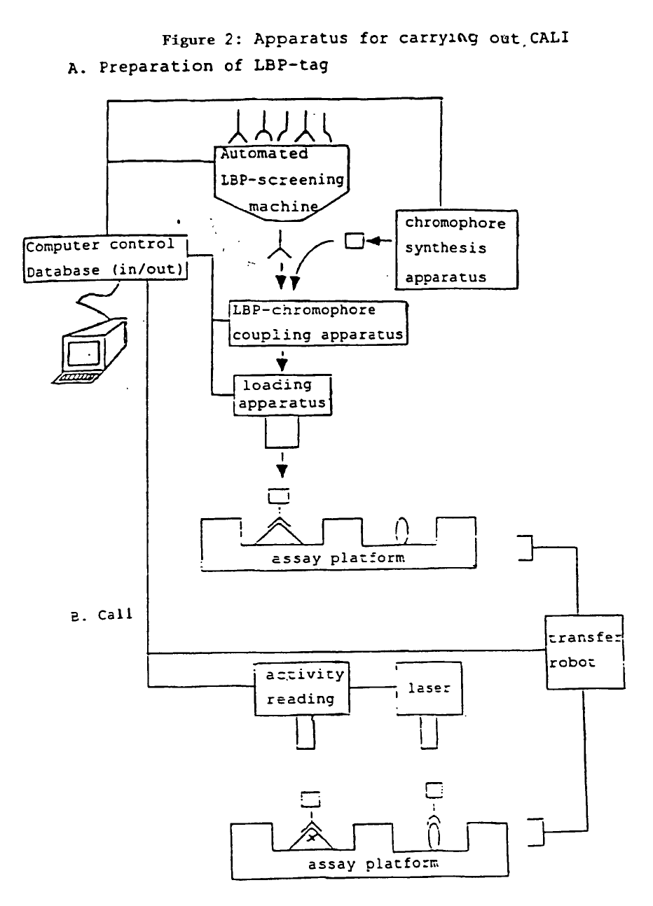

The invention also relates to an apparatus for carrying out the method according to the invention, depicted in detail in FIG. 2. This apparatus is an automated system consisting of integrated independent units/parts for identifying the protein function. Part A relates to the preparation of LBP-tag.

An automated LBP screening machine provides specific LBPs which are directed against specific target molecules/ligands, while the chromophore synthesis apparatus produces chromophores of choice. The selected LBPs and the synthesized chromophores are linked chemically in an LBP-chromophore coupling apparatus, resulting in LBP-tag. This LBP-tag is transferred into a loading apparatus which transfers the LBP-tag into predetermined cavities which are coated with the target molecule/ligand in the assay platform or contain same in solution or contain cells comprising said target molecule. A transfer robot then moves the assay platform into the laser system in order to initiate the second part B. The samples are irradiated with the laser at the required wavelength in order to induce a modification by free radicals. An apparatus for reading the activity then passes over the cavity which has been laser-irradiated in order to record the biological or chemical activity of the irradiated samples.

All the parts of the apparatus are connected to a central computer system for monitoring and analysis.

The invention is explained in detail by the following examples and figures which are provided for illustration of several embodiments of the invention and should not be interpreted as limiting the scope of the invention.

### EXAMPLE 1

### Selection of scFv Antibodies and CALI of β-galactosidase

First Set of Experiments:

i) Selection of anti-β-galactosidase scFv Antibodies by Phage Display.

A phage display single-chain antibody (scFv) library was produced as previously described (Sheets, M. D. et al. 1998, PNAS, 95:6157-6162; Marks, J. D. et al., 1991, J. Mol. Biol. 222:581-597). ScFv antibodies specific for β-galactosidase were isolated from this library by phage display and biopanning using methods previously described (Sheets, M. D. et al. 1998, PNAS, 95:6157-6162; Marks, J. D. et al., 1991, J. Mol. Biol. 222:581-597). The wells of an ELISA plate were coated with purified β-galactosidase and used for selection by phage display. After performing a panning, the enriched phages which contained an scFv with specificity for β-galactosidase were subcloned into an expression vector (Marks, J. D. et al., 1991, J. Mol. Biol. 222:581-597) and tested for specificity for β-galactosidase using an ELISA and by their ability to inhibit β-galactosidase activity (Wallenfells, K., 1962, Methods Enzymol. 5:212-219). An anti-testosterone scFv was used as negative control.

Several scFvs which did not inhibit β-galactosidase activity or had a similar β-galactosidase activity as the negative control were used for the next step.

ii) Labelling of the scFv Antibodies with Fluorescein and Malachite Green

The scFvs as obtained above (which do not inhibit β-galactosidase activity) against β-galactosidase were labelled with malachite green isothiocyanate or fluoroscein isothiocyanate with modifications as described (Jay, D. G. 1988, PNAS 85: 5454-5458; Surrey, T. 1998, PNAS 95:4293-4298). Stated briefly, antibodies in a concentration of 600 μg/ml in 500 mM NaHCO₃ (pH 9.8) were labelled stepwise by adding malachite green isothiocyanate or fluorescein isothiocyanate (from Molecular Probes, Eugene, Oreg.) up to a concentration of 120 μg/ml from a stock solution (20 mg/ml or 2 mg/ml) in DMSO. After incubation with stirring at room temperature for 1 hour or incubation on ice for 4 hours, the solution was passed through a desalting column in 150 mM NaCl/50 mM NaPi, pH 7.3 (for malachite green it is necessary to centrifuge the precipitate before changing the buffer) in order to remove the marker from the labelled protein. The labelled antibodies were isolated and tested for the presence of the chromophore by absorption.

iii) CALI of β-galactosidase

The use of the laser and the exposure to a laser beam for CALI takes place essentially as described (Beerman, A. E. and Jay, D. G. 1994, Methods Cell Biol. 44, 715-732; Surrey, T. 1998, PNAS 95:4293-4298). A sample which contained β-galactosidase and dye-labelled antibodies against β-galactosidase from ii) was placed on an ELISA plate. The total volume in the well was exposed to a laser beam for various periods of time. The activity of the samples was measured as described above. The negative controls consisted of an scFv chromophore/β-Gal complex which had not been laser irradiated, and of a CALI experiment with anti-testosterone scFv.

The beta-galactosidase activity after CALI was followed as a function of time (0, 1, 3, 5, 10, and 30 min.). The activity was measured as units which are appropriate for the test and are expressed as the ratio to the activity of the untreated β-galactosidase.

Second Set of Experiments:

i) Selection of anti-β-galactosidase scFv Antibodies by Phage Display.

A phage display single-chain antibody (scFv) library was produced as previously described (Sheets, M. D. et al. 1998, PNAS, 95:6157-6162; Marks, J. D. et al., 1991, J. Mol. Biol. 222:581-597). ScFv antibodies specific for β-galactosidase were isolated from this library by phage display and biopanning using methods previously described (Sheets, M. D. et al. 1998, PNAS, 95:6157-6162; Marks, J. D. et al., 1991, J. Mol. Biol. 222:581-597). Specifically, library construction and phage selection was performed as follows. Total RNA was extracted from spleens of mice immunized with β-galactosidase (250 μg/mouse). After reverse transcription of the mRNA, the genes for the immunoglobulin variable regions were amplified via PCR using a primer set of 22 specific primers which covers approximately 60-70% of the used mouse immune repertoire. The gene pools for the variable regions of light (VL) and heavy chains (VH) were then inserted successively into the phagemid vector pCANTAB 5E (Amersham Pharmacia), modified by insertion of a 15 amino acid glycine-rich linker (GGGGSGGGGSGGGGS; SEQ. ID. NO. 1) and two pairs of restriction sites (SfiI/SalI for VH, ApaLI/NotI for VL). The resulting vector pool—consisting of 5×10⁶ independent clones—was used for transformation of E. coli cells and phage particles were subsequently prepared by infection of the transformed cells with helper phage as described (Marks, J. D. et al. 1991, J. Mol. Biol. 222:581-597). The phage pool was then subjected to one round of panning against 10 μg/microtiter well (coating concentration) of purified β-galactosidase (Sigma). Unbound phages were removed by washing (six 5 min washes with PBS pH 7.4, 0.05% Tween 20, followed by six 5 min washes with PBS pH 7.4) and the bound phages eluted in a two-step procedure starting with a suspension of log-phase E. coli cells followed by a pH elution (10 mM glycine/HCl pH 2.2). Re-infected E. coli cells of both eluates were used for isolation of phagemid vector pools. The genes coding for the scFv antibodies were subcloned in the expression vector pUC119 in frame with peptide tags for detection (E-tag) and purification (His-tag) fused to the C-terminus of the scFv.

Individual scFv antibody clones were expressed in a 96-well format and cell lysates tested for antibody expression by dot blot and for binding to β-galactosidase by ELISA. Clones leading to a positive signal were sequenced and checked for differences in their antigen binding regions. The specific interaction of the scFv with β-galactosidase was confirmed by specificity ELISA in comparison with five other protein antigens.

For use in CALI-experiments single antibody clones or mixtures of clones were expressed in culture volumes ranging from 0.5-11. After harvesting, cells were lysed by addition of lysozyme and subsequent sedimentation of the cell debris. The recombinant scFv was then purified via the His-tag by immobilized metal affinity chromatography. For this, the lysate containing the recombinant scFv antibody was loaded onto a column filled with 1 ml TALON resin (Clontech) and the scFv subsequently eluted in 150 mM imidazole. The eluates were subjected to gelfiltration (Sephadex 200 column, Amersham) for removal of imidazole and further purification.

ii) Labeling of scFv and Whole Antibodies

Antibodies were dialyzed against PBS buffer (pH 7.4) over night. After dialysis, scFvs were concentrated to 100-500 μg/ml with ultrafiltration spin columns (YM-10, Millipore) prior to labeling with fluorescein isothyocyanate (FITC, Molecular Probes) in typically 100 μl. The pH was adjusted to basic conditions (pH 9.0-9.5) with 20 μl NaHCO₃-buffer (0.5 M; pH 9.5). The amount of FITC-solution in DMSO (10 mg/ml) added to the labeling reaction was calculated according to the supplier's manual (Molecular Probes). The molar ratio of FITC to antibodies was 10:1. The solution was incubated for 2.5 h at room temperature (protected from light) being constantly mixed by inversion. After the labeling reaction, unbound dye was separated from labeled antibodies by gelfiltration with 5 min centrifugation at 1000×g through sepharose spin columns (Bio-Gel P6, Bio-Rad). Protein and dye concentrations were calculated from OD readings at 280 nm and 494 nm, respectively. From this, the antibody labeling ratios were calculated according to the supplier's manual (Molecular Probes). These conditions can principally also be used for labeling with malachite green.

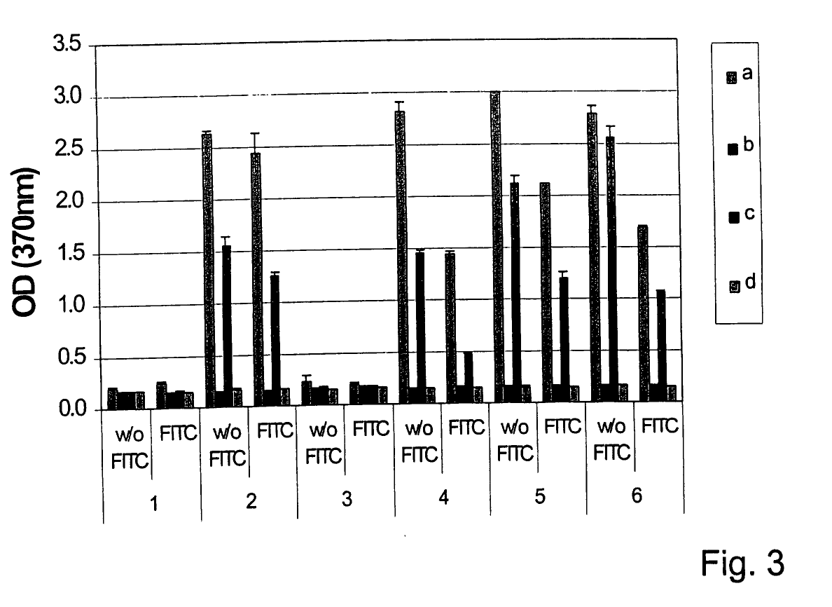

The binding of FITC-labeled and unlabeled scFv to β-galactosidase was tested by Elisa as shown in FIG. 3. The labeled scFv retained at least 50% of the binding activity of unlabeled scFv. 1, unselected anti-β-galactosidase library; 2, scFv pool after one round of selection against β-galactosidase; 3, non-binding scFv 1A1 as negative control; 4, anti-β-galactosidase scFv 1A4; 5, pool of 20 anti-β-galactosidase scFv; 6, anti-β-galactosidase scFv 3A6. a, scFv used at 1 μg/ml on wells coated with 50 nM β-galactosidase; b, scFv used at 1 μg/ml on wells coated with 2.5% low fat milk; c, scFv used at 0.1 μg/ml on wells coated with 50 nM β-galactosidase; d, scFv used at 0.1 μg/ml on wells coated with 2.5% low fat milk. The Elisa was developed with a secondary, horseradish peroxidase-coupled anti-E tag antibody and colorimetric POD substrate (Roche Diagnostics)

iii) CALI of β-galactosidase Using scFv and Whole Antibodies

CALI of β-galactosidase as well as determination of β-galactosidase activity was performed, with modifications, essentially as described (Beerman, A. E. and Jay, D. G. 1994, Methods Cell Biol. 44, 715-732; Surrey, T. 1998, PNAS 95:4293-4298; Wallenfells, K. 1962, Methods Enzymol. 5:212-219). Specifically, a mix of β-galactosidase (400 ng/ml), FITC-labeled antibodies (10 μg/ml) and BSA (250 μg/ml) in PBS (pH 7.4), was incubated for 1 h at room temperature (protected from light). 50 μl aliquots were pipetted into a flat bottom 96-well micro titerplate. Samples were irradiated at 494 nm or not irradiated. Irradiation was performed with a tunable Nd:YAG laser (Infinity-XPO, Coherent) at a pulse frequency of 30 Hz. Each well was subjected to 5 min of laser irradiation at 7.5 mJ per pulse with a beam adjusted to the size of the well. The activity of β-galactosidase in irradiated and non-irradiated samples was subsequently determined by adding 50 μl/well of 4 mg/ml ONPG (o-Nitrophenyl-β-D-galactopyranoside). After 30 min incubation at 37° C., the reaction was stopped by addition of 90 μl of Na₂CO₃ (1M) and the absorbance change resulting from ONPG conversion was measured in a plate reader at 405 nm.

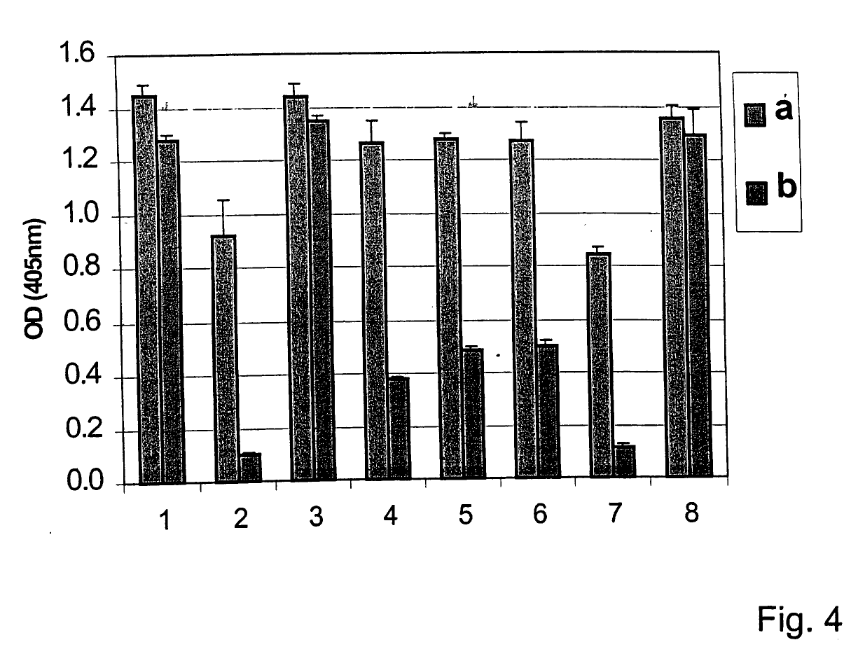

FIG. 4 shows the degree of inactivation of β-galactosidase by CALI with FITC using different scFv and whole antibodies. Over 90% selective inactivation of enzyme activity was achieved with polyclonal antibody pools (2 and 7) and 65-75% with monoclonal scFv (4 and 6). All antibodies were FITC-labeled and used at 20 μg/ml. Similar results were obtaine with malachite green-labeled antibodies. 1, unselected anti-β-galactosidase library; 2, scFv pool after one round of selection against β-galactosidase; 3, non-binding scFv 1A1 as negative control; 4, anti-β-galactosidase scFv 1A4; 5, pool of 20 anti-β-galactosidase scFv; 6, anti-β-galactosidase scFv 3A6; 7, polyclonal rabbit anti-β-galactosidase IgG; 8, non-immune rabbit IgG; a, non-irradiated; b, irradiated 5 min at 494 nm.

### EXAMPLE 2

### Selection of Aptamers and CALI of D-galactosidase

i) Selection of the aptamers specific for β-galactosidase by SELEX

(systematic evolution of ligands by exponential enrichment) according to a previously described method (Morris, K. N. et al. 1998, PNAS 95:2902-2907).

ii) Labelling of nucleotide aptamers with fluorescein and malachite green according to previously described methods (Igloi, G. L. 1996, Anal. Biochem. 233:124-129; Meyer and Hanna, 1996, Bioconjug. Chem. 4:401-412; Nelson, P. S. et al. 1992, Nucleic Acids Res. 20:6253-6259; Thiesen, P. et al. 1992, Nucleic Acids Symp. Ser 27:99-100; Proudnikov, D. & Mizabekov, A. 1996, Nucleic Acids Res. 24:4535-4542; Rosemeyer, V. et al. 1995, Anal. Biochem. 224:446-449; Richardson, R. W. & Gumport, R. I. 1983, Nucleic Acids Res. 11:6167-6184; Kinoshita, Y. et al. 1997, Nucleic Acids Res. 25:3747-3748).

iii) CALI of β-galactosidase was performed principally as described before (Beerman, A. E. and Jay, D. G. 1994, Methods Cell Biol. 44, 715-732; Surrey, T. 1998, PNAS 95:4293-4298).

### EXAMPLE 3

### Production of Genetically Modified scFv with Preferential Labelling Sites

First Set of Experiments:

In order to avoid the restriction of unpredicatable labelling of scFv with the chromophore, a sequence of Lys residues was introduced by genetic manipulation into the linker region of the scFv. Standard gene synthesis methods known to a person skilled in the field are used for this (Prodromou, C. & Pearl, L. H. 1992, Prot. Eng. 5: 827-829). After the labelling with the chromophore as described in Example 1 (ii), the presence of the chromophore was detected and determined quantitatively by absorption as described above. In addition, the recombinant scFvs labelled with the chromophore were tested for binding to β-Gal by ELISA as described above.

The scFvs produced by genetic manipulation and labelled with the chromophore retained their ability to bind to β-Gal.

Second Set of Experiments:

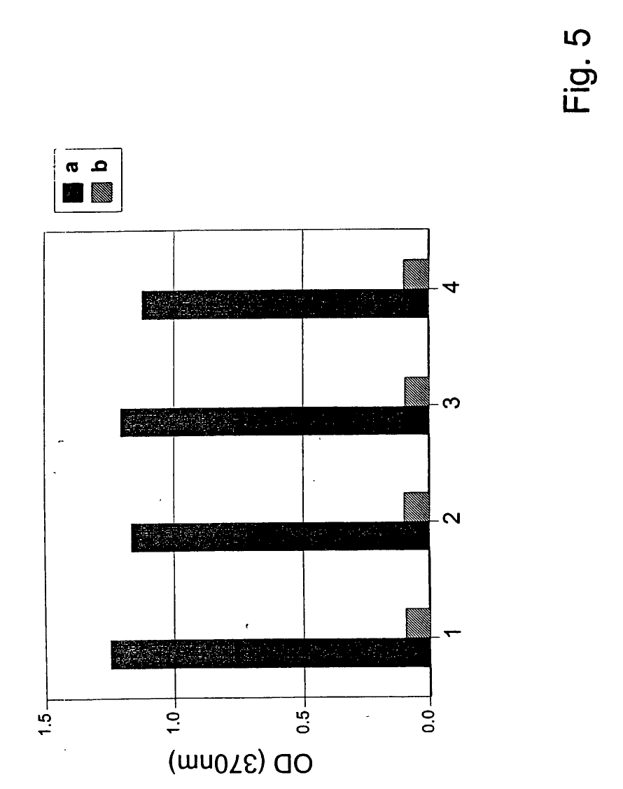

In order to minimize the restriction of unpredicatable labelling of different scFv with chromophores/fluorophores, a sequence of Lys residues was introduced by genetic manipulation in the shortened linker region of a scFv to obtain a diabody version of the scFv with a lysine-rich, accessible linker (GKGGKGGKS; SEQ. ID. NO. 2). Standard gene synthesis methods known to a person skilled in the field are used for this (Prodromou, C. & Pearl, L. H. 1992, Prot. Eng. 5: 827-829). A FITC-labeled and unlabeled scFv and its lysine-rich diabody version were tested for specific binding to β-galactosidase by ELISA as shown in FIG. 5. All antibodies were used at 12.5 μg/ml. 1, unlabeled scFv 1A4; 2, FITC-labeled scFv 1A4; 3, unlabeled, lysine-rich diabody 1A4; 4, FITC-labeled, lysine-rich diabody 1A4. a, wells coated with 50 nM β-galactosidase; b, wells coated with 2.5% low fat milk. The Elisa was developed with a secondary, horseradish peroxidase-coupled anti-E tag antibody and colorimetric POD substrate (Roche Diagnostics).

The FITC-labeled and unlabeled, lysine-rich diabody showed virtually identical binding activities (3 and 4) to β-galactosidase which were also indistinguishable from binding activity in its scFv state (1 and 2). Thus, lysine-rich scFv diabodies are suitable for use in CALI experiments.

It will be apparent to those skilled in the art that various modifications and variations can be made to the processes and apparatus of the invention. Thus, it is intended that the present invention covers such modifications and variations, provided they come within the scope of the appended claims and their equivalents.

What is claimed is:

1) A method for identifying the function of a ligand L using chromophore-assisted laser inactivation (CALI), characterized by the steps of:
a) selecting a ligand binding partner (LBP) with specificity for the ligand L,
b) coupling the LBP to a laser-activatable marker (tag) to form LBP-tag,
c) contacting L with said LBP-tag to form an L/LBP-tag complex, and
d) irradiating the L/LBP-tag complex with a laser beam, whereupon the irradiated LBP-tag selectively modifies the bound ligand, it being possible to interchange the sequence of stages b) and c).

2) The method according to claim 1, characterized in that the LBP is selected from the group consisting of dsFv (disulfide-linked variable chain fragment), scFv (single chain variable chain fragment), Fab (fragment, antigen-binding), diabody, immunoglobulin-like molecules, peptides, RNA, DNA, PNA, and small organic molecules, except intact antibody molecules.

3) The method according to claim 1, characterized in that the LBP is derived from a combinatorial library, with the exception of whole antibody LBPs derived from cell fusion hybridoma technology.

4) The method according to claim 1, characterized in that the laser-activatable marker is selected from the group consisting of malachite green, fluorescein, lissamine rhodamine, tetramethylrhodamine isothiocyanate, cyanin 3.18, AMCA-SE (7-amino-4-methylcoumarin-3-acetic acid, succinimidyl ester), AMCA (7-amino-4-methylcoumarin-3-acetic acid), BODIPY® (4,4-difluoro-4-bora-3a,4a-diaza-S-indacene) and variants thereof, Cascade Blue, Cl-NERF, dansyl, dialkylamino-coumarin,4′,5′-dichloro-2′,7′-dimethyoxyfluorescein, DM-NERF, eosin, eosin F3S, erythrosin, hydroxycoumarin, Isosulfan Blue, lissamine rhodamine B, malachite green, methoxycoumarin, naphthofluorescein, NBD, Oregon Green 488, 500, 514, PyMPO (4-(5-(4-methoxyphenyl)oxazol-2-yl)pyridine), pyrene, Rhodamine 6G, Rhodamine Green, Rhodamine Red, Rhodol Green, 2′,4′,5′,7′-tetrabromosulphonefluorescein, tetramethylrhodamine, Texas Red and X-rhodamine.

5) The method according to claim 1, characterized in that the LBP is modified by attaching or genetically introducing lysine residues.

6) The method according to claim 1, characterized in that the LBP is modified by genetic engineering fusion or chemical coupling to peptides or polypeptides to allow efficient transport of the LBP-Tag into cells.

7) The method of claim 1, wherein said identified ligand L is used for the development a drug for the treatment of a disease.

8) An apparatus for carrying out a method according to claim 1, characterized in that it is an automated system consisting of integrated independent units/parts for identifying the protein function and comprises the following constituents:
(a) an automated LBP screening machine for producing specific LBPs which are directed against specific target molecules/ligands,
(b) a chromophore synthesis apparatus for producing chromophores,
(c) an LBP-chromophore coupling apparatus for linking the selected LBPs and the synthesized chromophores,
(d) a loading apparatus for transferring the LBP-tag into predetermined cavities which are coated with the target molecule/ligand in the assay platform, or contain same in solution or contain cells comprising said target molecule,
(e) a transfer robot arm for moving the assay platform into the laser system,
(f) an apparatus for reading the activity,
(g) a database,
(h) a central computer system.


SEQUENCE LISTING (2 sequences)

SEQ ID NO: 1 - protein, 15 aa, Artificial Sequence
  Synthetic peptide
GGGGSGGGGSGGGGS

SEQ ID NO: 2 - protein, 9 aa, Artificial Sequence
  Synthetic peptide
GKGGKGGKS